(19) United States
(12) Patent Application Publication
(10) Pub. No.: US 2023/0051666 A1
(43) Pub. Date: Feb. 16, 2023

(54) Medical Device Packaging

(71) Applicant: Boston Scientific Medical Device Limited, Galway (IE)

(72) Inventor: Vinayak Patil, Ambegaon (IN)

(73) Assignee: Boston Scientific Medical Device Limited, Galway (IE)

(21) Appl. No.: 17/886,183

(22) Filed: Aug. 11, 2022

Related U.S. Application Data
(60) Provisional application No. 63/231,915, filed on Aug. 11, 2021.

(51) Int. Cl. A61M 25/00

(52) U.S. Cl. CPC A61M 25/002; A61M 2207/10

(57) ABSTRACT

Medical device packaging and methods for shaping/treating medical devices are disclosed. An example medical device package may include a carrier tube having a first end and a second end. The carrier tube may have an outer diameter adjacent to the first end. An intermediate tube may be coupled to the first end. The intermediate tube may have inner diameter larger than the outer diameter of the carrier tube. A retractable tube may be coupled to the intermediate tube. A clip may be secured to the carrier tube, the intermediate tube, or both.

### CROSS-REFERENCE TO RELATED APPLICATIONS

This application claims the benefit of priority under 35 U.S.C. § 119 of U.S. Provisional Application No 63/231,915, filed Aug. 11, 2021, the entire disclosure of which is hereby incorporated by reference.

### TECHNICAL FIELD

The present disclosure pertains to medical devices, and methods for manufacturing medical devices. More particularly, the present disclosure pertains to medical device packaging.

### BACKGROUND

A wide variety of intracorporeal medical devices have been developed for medical use, for example, intravascular use. Some of these devices include guidewires, catheters, and the like. These devices are manufactured by any one of a variety of different manufacturing methods, may be used according to any one of a variety of methods, and may be packaged in a number of different packages. Of the known medical devices, methods, and packages, each has certain advantages and disadvantages. There is an ongoing need to provide alternative medical devices, alternative methods for manufacturing and using medical devices, and alternative packages for medical devices.

### BRIEF SUMMARY

This disclosure provides design, material, manufacturing method, and use alternatives for medical devices. A medical device package is disclosed. The medical device package comprises: a carrier tube having a first end and a second end, the carrier tube having an outer diameter adjacent to the first end; an intermediate tube coupled to the first end, the intermediate tube having an inner diameter larger than the outer diameter the carrier tube; a retractable tube coupled to the intermediate tube; and a clip secured to the carrier tube, the intermediate tube, or both.

Alternatively or additionally to any of the embodiments above, the retractable tube has a second outer diameter smaller than the inner diameter of the intermediate tube.

Alternatively or additionally to any of the embodiments above, the second outer diameter is substantially the same as the outer diameter of the carrier tube.

Alternatively or additionally to any of the embodiments above, the intermediate tube is secured to the carrier tube by the clip.

Alternatively or additionally to any of the embodiments above, the intermediate tube is frictionally engaged with the carrier tube.

Alternatively or additionally to any of the embodiments above, the intermediate tube is bonded with the carrier tube.

Alternatively or additionally to any of the embodiments above, the retractable tube is slidable within the intermediate tube.

Alternatively or additionally to any of the embodiments above, further comprising a medical device disposed within the carrier tube.

Alternatively or additionally to any of the embodiments above, further comprising a mandrel disposed within a distal end region of the medical device.

Alternatively or additionally to any of the embodiments above, the mandrel includes a grasping loop region.

A medical device package is disclosed. The medical device package comprises: a carrier tube having an end region; an intermediate tube disposed over the end region and extending distally from the carrier tube; a clip secured to the intermediate tube; a retractable tube slidably disposed relative to the intermediate tube, the retractable tube being configured to shift between a first position where a distal end of the retractable tube is disposed adjacent to a distal end of the intermediate tube and a second position where the distal end of the retractable tube is extended distally from the distal end of the intermediate tube; and a medical device disposed within the carrier tube, the intermediate tube, and the retractable tube.

Alternatively or additionally to any of the embodiments above, the retractable tube is slidably disposed within the intermediate tube.

Alternatively or additionally to any of the embodiments above, further comprising a mandrel disposed within a distal end region of the medical device.

Alternatively or additionally to any of the embodiments above, the mandrel includes a grasping loop region.

A method for shaping a portion of a medical device is disclosed. The method comprises: actuating a retractable tube assembly to expose an exposable portion of a medical device disposed within a carrier tube; wherein the retractable tube assembly includes an intermediate tube coupled to a distal end of the carrier tube and a retractable tube coupled to a distal end of the intermediate tube; shaping the exposable portion of the medical device into a desired shape; and actuating the retractable tube assembly to cover the exposable portion of the medical device.

Alternatively or additionally to any of the embodiments above, the carrier tube has an outer diameter and the intermediate tube has an inner diameter bigger than the outer diameter of the carrier tube.

Alternatively or additionally to any of the embodiments above, the retractable tube has a second outer diameter that is smaller than the inner diameter of the intermediate tube.

Alternatively or additionally to any of the embodiments above, actuating a retractable tube assembly to expose an exposable portion of a medical device disposed within a carrier tube includes shifting the retractable tube proximally within the intermediate tube.

Alternatively or additionally to any of the embodiments above, actuating the retractable tube assembly to cover the exposable portion of the medical device includes distally shifting the retractable tube relative to the intermediate tube.

Alternatively or additionally to any of the embodiments above, further comprising disposing a mandrel within a distal end region of the medical device.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example medical device package with a medical device therein.

FIG. 2 is a side view of an example medical device package.

FIG. 3 is a side view of a portion of an example medical device package.

FIG. 4 is a side view of a portion of an example medical device package.

FIG. 5 is a side view of a portion of an example medical device package.

FIGS. 6-10 depict a use of an example medical device package.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

The manufacturing and packaging of medical devices can include a number of different processes. Some processes may include shaping processes where the distal end region of a medical device can be curved. In such processes, the medical device may be disposed in a carrier tube and the distal end region of the medical device is exposed and/or otherwise extends out from carrier tube in order to facilitate shaping. Once shaped, the distal end of the medical device can be protected with a covering such as a clamshell tray. Disclosed herein are medical device packages that can provide a desirable amount of protection for medical devices and also may help to facilitate processes such as shaping processes.

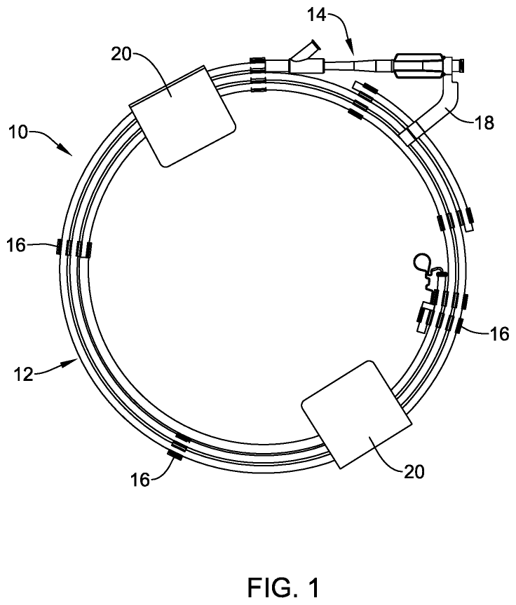

FIG. 1 illustrates an example medical device package 10. The package 10 includes a hoop structure or carrier tube 12. A medical device 14 may be disposed within the carrier tube 12. In general, the carrier tube 12 may be wound into a spiral configuration. Adjacent windings of the carrier tube 12 may be secured using one or more clips 16. In some instances, an angled clip 18 may secure the medical device 14. One or more instruction panels 20 may also be secured to the carrier tube 12. The instruction panels 20 may take the form of stickers that may also help to hold together the windings of the carrier tube 12.

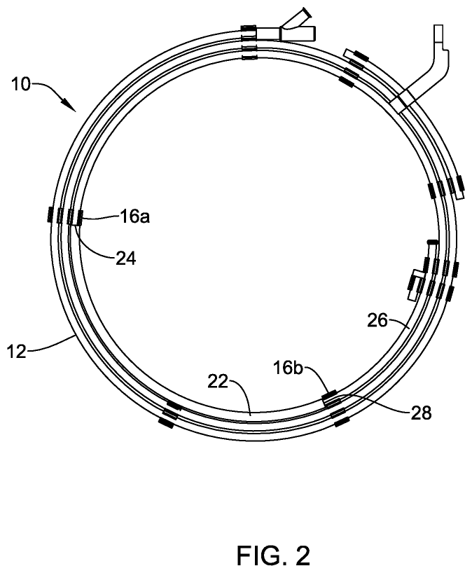

FIG. 2 illustrates the package 10 prior to the medical device 14 being loaded therein (and/or with the medical device 14 removed). Here it can be seen that one or more tubes may be coupled to the carrier tube 12. For example, the carrier tube 12 may have a first end and a second end. An intermediate tube 22 may be disposed at a first or distal end 24 of the carrier tube 12. In some instances, the intermediate tube 22 may be bonded to the carrier tube 12 using a suitable bond such as an adhesive bond, a thermal bond, a mechanical bond, a frictional bond, etc. In some of these and in other instances, the intermediate tube 22 may be secured to the carrier tube 12 by one of the clips (e.g., clip 16a as shown in FIG. 2). A retractable tube 26 may be disposed at a distal end 28 of the intermediate tube 22. In some instances, the retractable tube 26 may be secured to the intermediate tube 22 by one of the clips (e.g., clip 16b as shown in FIG. 2).

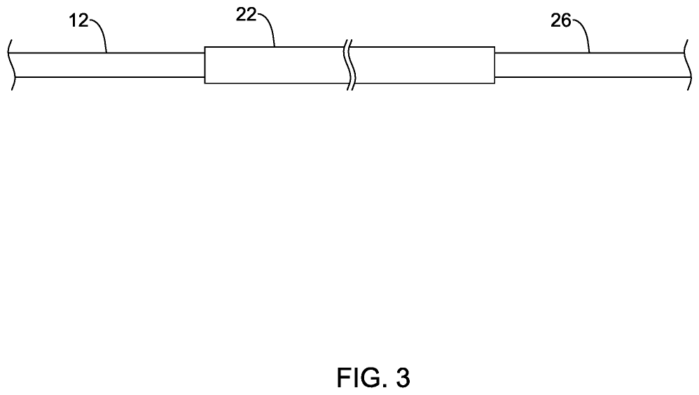

The intermediate tube 22 and the retractable tube 26 may help to facilitate processes such as tip shaping processes. For example, the intermediate tube 22 may have an outer diameter that is larger than the outer diameter of the carrier tube 12 as shown in FIG. 3. For example, the carrier tube 12 may have an outer diameter of about 0.140-0160 inches or about 0.150-0.154 inches, or about 0.152 inches. The intermediate tube 22 may have an outer diameter of about 0.145-0165 inches or about 0.154-0.160 inches, or about 0.157 inches. In some instances, the intermediate tube 22 may have an inner diameter sized to accommodate the carrier tube 12 therein (e.g., the inner diameter of the intermediate tube 22 may be larger than the outer diameter of the carrier tube 12). In other words, the carrier tube 12 (e.g., the distal end region of the carrier tube 12) may be disposed within the intermediate tube 22. The intermediate tube 22 may also have an outer diameter that is larger than the outer diameter of the retractable tube 26. In some instances, the intermediate tube 22 may have an inner diameter sized to accommodate the retractable tube 26 therein (e.g., the inner diameter of the intermediate tube 22 may be larger than the outer diameter of the retractable tube 26). In other words, the retractable tube 26 (e.g., the proximal end region of the retractable tube 26) may be disposed within the intermediate tube 22. In some instances, the inner and/or outer diameter of the retractable tube 26 may be substantially the same as the inner and/or outer diameter of the carrier tube 12. Other configurations are contemplated.

The arrangement of the carrier tube 12, the intermediate tube 22, and the retractable tube 26 allows the retractable tube 26 to be proximally retracted in order to uncover or otherwise expose a portion (e.g., a distal end region) of a medical device disposed therein (e.g., the medical device 14). For example, by virtue of the retractable tube 26 being disposed within the intermediate tube, the retractable tube 26 may be able to be slid proximally within the intermediate tube 22. This may include shifting the retractable tube 26 between a first position where a distal end of the retractable tube 26 is disposed adjacent to the distal end 28 of the intermediate tube 22 and a second position where the distal end of the retractable tube 26 is extended distally from the distal end 28 of the intermediate tube 22. In some instances, the sliding of the retractable tube 26 may be described as telescoping or telescopic.

The length that the retractable tube 26 may be slid can be controlled by configuring the lengths of the intermediate tube 22 and/or the retractable tube 26. For example, the retractable tube 26 may be proximally retracted within the intermediate tube 22 until the proximal end of the retractable tube 26 engages the carrier tube 12. Accordingly, adjusting the length of the intermediate tube 22 may allow for the length of the retractable tube 26 that is retracted to be adjusted.

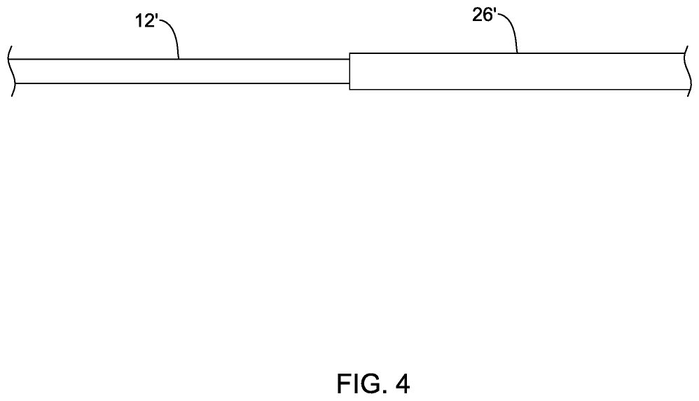

While the use of the carrier tube 12, a larger intermediate tube 22, and a smaller retractable tube 26 (e.g., a “three tube configuration” or “three part configuration” that includes the carrier tube 12 and two additional tubes) allows for suitable sliding action (e.g., that allows the distal end region of a medical device to be uncovered), other arrangements are possible. For example, FIG. 4 illustrates another example configuration where the retractable tube 26′ may be coupled to the carrier tube 12′. In this example, the retractable tube 26′ has an outer diameter that is larger than the carrier tube 12′. The retractable tube 26′ may have an inner diameter sized to accommodate or otherwise fit over the carrier tube 12′ so that the retractable tube 26′ can be proximally slid along the exterior of the carrier tube 12′. In this example, the intermediate tube may not be necessary. Such an arrangement may be an example of a “two tube configuration” or “two part configuration” that includes the carrier tube 12′ and one additional tube.

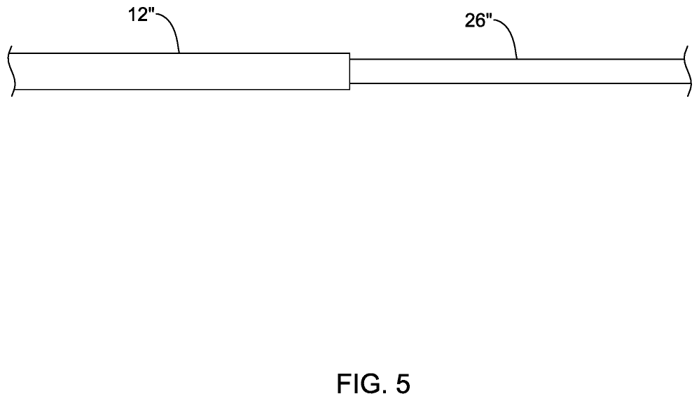

Alternatively, FIG. 5 illustrates another example configuration where the retractable tube 26″ may be coupled to the carrier tube 12″. In this example, the retractable tube 26″ has an outer diameter that is smaller than the carrier tube 12″. The carrier tube 12″ may have an inner diameter sized to accommodate the retractable tube 26″ therein so that the retractable tube 26″ may be slid proximally within the carrier tube 12″. In this example, the intermediate tube may not be necessary. Such an arrangement may be an example of a “two tube configuration”.

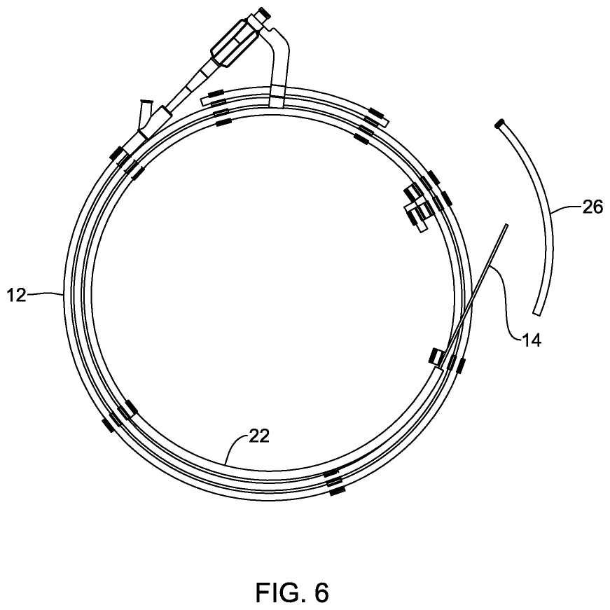

FIGS. 6-10 illustrate an example use and/or process such as a tip shaping process. For example, FIG. 6 illustrates the medical device 14 loaded into the carrier tube 12. In this example, the retractable tube 26 is shown disconnected from the intermediate tube 22. Upon loading the medical device 14 into the carrier tube 12, the retractable tube 26 may be coupled to the intermediate tube 22 (e.g., and be arranged in a manner similar to what is shown in FIG. 2).

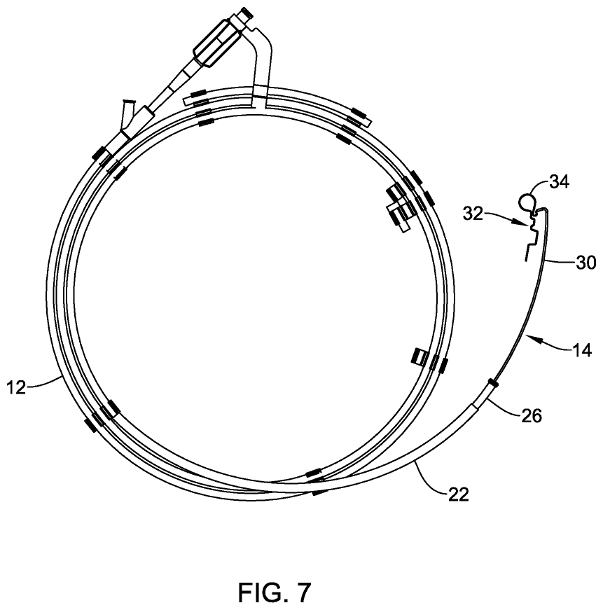

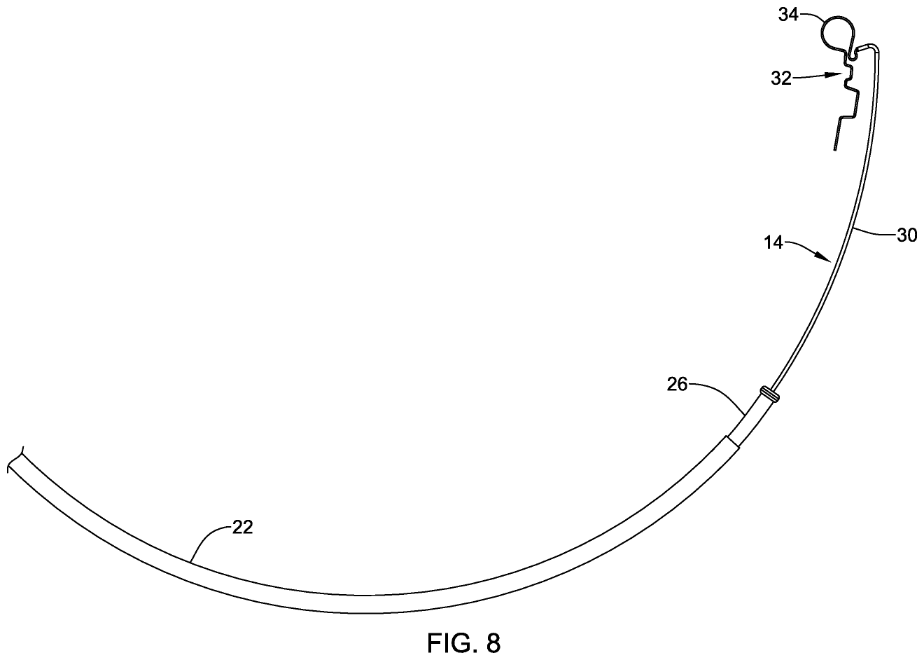

In some instances, it may be desirable to process the medical device 14. For example, it may be desirable to use a shaping process to shape the distal end region of the medical device 14. To facilitate this process, the retractable tube 26 may be proximally retracted into the intermediate tube 22 to expose a region 30 (e.g., a distal end region 30) of the medical device 14 as depicted in FIGS. 7-8. The exposed distal end region 30 may then undergo processing (e.g., a shaping process). Prior to or after the shaping process, a mandrel 32 may be disposed within the distal end region 30 of the medical device 14. The mandrel 32 may help to protect the medical device 14. In some instances, the mandrel 32 may include a grasping loop region 34 that may help to facilitate removal of the mandrel 32.

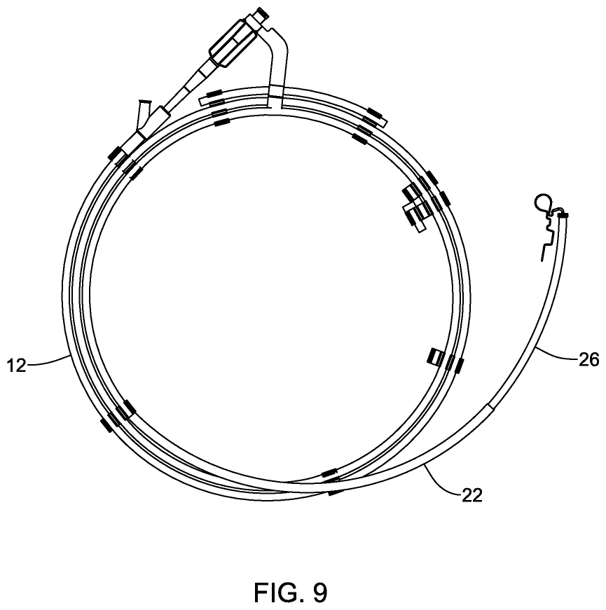

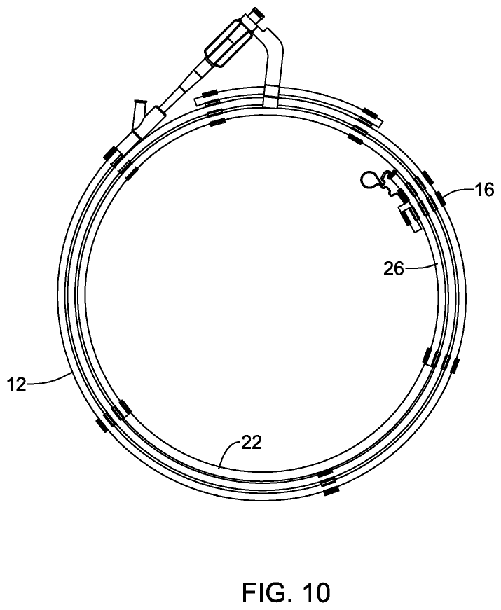

Upon completion of the shaping process, the retractable tube 26 may be distally advanced to re-cover the distal end region 30 of the medical device 14 as shown in FIG. 9. The retractable tube 26 may be fitted into the clip 16 as shown in FIG. 10. In some instances, the packaged medical device 14 (e.g., the medical device 14 within the package 10) may be disposed in a suitable pouch and may undergo a suitable sterilization process.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device package, comprising:
a carrier tube having a first end and a second end, the carrier tube having an outer diameter adjacent to the first end;
an intermediate tube coupled to the first end, the intermediate tube having an inner diameter larger than the outer diameter the carrier tube;
a retractable tube coupled to the intermediate tube; and
a clip secured to the carrier tube, the intermediate tube, or both.

2. The medical device package of claim 1, wherein the retractable tube has a second outer diameter smaller than the inner diameter of the intermediate tube.

3. The medical device package of claim 2, wherein the second outer diameter is substantially the same as the outer diameter of the carrier tube.

4. The medical device package of claim 1, wherein the intermediate tube is secured to the carrier tube by the clip.

5. The medical device package of claim 1, wherein the intermediate tube is frictionally engaged with the carrier tube.

6. The medical device package of claim 1, wherein the intermediate tube is bonded with the carrier tube.

7. The medical device package of claim 1, wherein the retractable tube is slidable within the intermediate tube.

8. The medical device package of claim 1, further comprising a medical device disposed within the carrier tube.

9. The medical device package of claim 8, further comprising a mandrel disposed within a distal end region of the medical device.

10. The medical device package of claim 9, wherein the mandrel includes a grasping loop region.

11. A medical device package, comprising:
a carrier tube having an end region;
an intermediate tube disposed over the end region and extending distally from the carrier tube;
a clip secured to the intermediate tube;
a retractable tube slidably disposed relative to the intermediate tube, the retractable tube being configured to shift between a first position where a distal end of the retractable tube is disposed adjacent to a distal end of the intermediate tube and a second position where the distal end of the retractable tube is extended distally from the distal end of the intermediate tube; and
a medical device disposed within the carrier tube, the intermediate tube, and the retractable tube.

12. The medical device package of claim 11, wherein the retractable tube is slidably disposed within the intermediate tube.

13. The medical device package of claim 11, further comprising a mandrel disposed within a distal end region of the medical device.

14. The medical device package of claim 13, wherein the mandrel includes a grasping loop region.

15. A method for shaping a portion of a medical device, the method comprising:
actuating a retractable tube assembly to expose an exposable portion of a medical device disposed within a carrier tube;
wherein the retractable tube assembly includes an intermediate tube coupled to a distal end of the carrier tube and a retractable tube coupled to a distal end of the intermediate tube;
shaping the exposable portion of the medical device into a desired shape; and
actuating the retractable tube assembly to cover the exposable portion of the medical device.

16. The method of claim 15, wherein the carrier tube has an outer diameter and the intermediate tube has an inner diameter bigger than the outer diameter of the carrier tube.

17. The method of claim 16, wherein the retractable tube has a second outer diameter that is smaller than the inner diameter of the intermediate tube.

18. The method of claim 17, wherein actuating a retractable tube assembly to expose an exposable portion of a medical device disposed within a carrier tube includes shifting the retractable tube proximally within the intermediate tube.

19. The method of claim 18, wherein actuating the retractable tube assembly to cover the exposable portion of the medical device includes distally shifting the retractable tube relative to the intermediate tube.

20. The method of claim 15, further comprising disposing a mandrel within a distal end region of the medical device.
